(19) United States
(12) Patent Application Publication
(10) Pub. No.: US 2025/0332022 A1
(43) Pub. Date: Oct. 30, 2025

(54) Menstrual cup with replaceable absorbent liner

(71) Applicant: Chante Knox, Atlanta, GA (US)

(72) Inventor: Chante Knox, Atlanta, GA (US)

(21) Appl. No.: 18/644,482

(22) Filed: Apr. 24, 2024

(51) Int. Cl. A61F 5/455; A61F 13/20

(52) U.S. Cl. CPC A61F 5/4553; A61F 13/2045

(57) ABSTRACT

The menstrual cup with replaceable absorbent liner includes a reusable structure that supports and surrounds replaceable, disposable liners. The menstrual cup with replaceable absorbent liner combines the best elements of reusable menstrual cups and disposable menstrual cups: the reusable outer shell is thicker and more resilient than a material designed for only single use, providing the outer shell with the structure and resilience necessary to hold the internal disposable absorbent members in place during use. Multiple internal structures to hold the absorbent pad against the reusable outer structure are disclosed. The reusable outer structure is flexible, allowing it to match the shape of the flexible absorbent pad and flexible rim.

### FIELD

This invention relates to the field of feminine hygiene products, and specifically to devices that collect menstrual fluid.

### BACKGROUND

Devices for absorbing menstrual fluid are known in the art. Each device seeks to solve the issue of discreetly collecting menstrual fluid. But known devices present significant drawbacks.

Sanitary napkins are bulky and prone to leakage. Tampons are less bulky but due to direct contact with the inner surface of the vaginal canal, tampons absorb all vaginal fluids, rather than being restricted to menstrual fluid. This complete absorption affects the natural moisture balance within the vaginal canal and can cause toxic shock syndrome.

What is needed is device that provides for the sanitary disposal of menstrual fluid without creating the risks and complications of existing devices.

### SUMMARY

The menstrual cup with replaceable absorbent liner includes a reusable structure that supports and surrounds replaceable, disposable liners. The result is a partially-reusable menstrual cup.

Existing reusable menstrual cups lack absorbent elements. As a result, when withdrawn from the vagina, the menstrual cup contains pool of fluids, including blood. After removal, the user must carefully dispose of the fluids while trying to avoid spilling and staining.

Disposable menstrual cups are made with and without absorbing elements. Without absorbing elements, disposable menstrual cups provide the same drawbacks as reusable menstrual cups. With absorbent elements, disposable menstrual cups result in an increased of amount of material that ends up in landfills.

The menstrual cup with replaceable absorbent liner combines the best elements of reusable menstrual cups and disposable menstrual cups: the reusable outer shell is thicker and more resilient than a material designed for only single use, providing the outer shell with the structure and resilience necessary to hold the internal disposable absorbent members in place during use.

Multiple internal structures, or internal retaining mechanisms, to hold the absorbent pad against the reusable outer structure are disclosed. The reusable outer structure is flexible, allowing it to match the shape of the flexible absorbent pad and flexible rim.

The first disclosed structure, or internal retaining mechanism, uses flexible internal walls with access through a central access opening or slit. The flexible internal walls can be moved upward, or popped upward, by the user's finger reaching into this central slit and pulling up. Because of the shape of the flexible internal walls, their shape at rest is convex or concave. Therefore, the flexible internal walls will snap between the two positions.

The internal retaining mechanism is optionally coupled to the reservoir.

When the flexible internal walls are in an open position, the absorbent liner is accessible and can be replaced. The flexible internal walls are then pushed, or popped back into place, where they hold the absorbent liner against the external structure of the menstrual cup.

The flexible internal walls include perforations in the forms of holes or slits to allow the menstrual fluid to pass through to the absorbent layer within.

The second disclosed structure uses flexible individual protrusions, or fingers, that extend over the internal surface of the absorbent member. The absorbent member is then held in place against the external structure of the menstrual cup. The flexible protrusions are readily displaced by the user, allowing access to the absorbent lining, allowing for its removal and replacement.

The external structure of the cup is consistent between embodiments. The upper circular rim or lip supports a downward projecting reservoir that is substantially conically shaped, or frustoconical with a rounded tip. Restated, the reservoir is defined by a rim and an external wall.

The reservoir includes an impermeable outer layer to contain menstrual fluid within the device. The non-absorbent outer layer also prevents the device from absorbing other fluids present within the vagina, and therefore reduces the risk of toxic shock syndrome.

The absorbent pad is preferably made from a material such as cotton. In alternative embodiments, the absorbent pad is made of an absorbent sponge similar to the sponges used for vaginal and nasal surgeries, such as a combination of chitosan hydrochloride and cellulose. The absorbent layer is held against an interior surface of the external wall by the internal retaining mechanism.

In the preferred embodiment, the absorbent pads are designed for single use and disposed of after use. In alternative embodiments, the absorbent pad is removable, to be washed, sanitized, dried, and replaced.

The absorbent pad can be a single conical or frustoconical element, or formed from two or more sections to simplify access and replacement by the user.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates an isometric view of the first embodiment in a use position of the menstrual cup with replaceable absorbent liner.

FIG. 2 illustrates an isometric view of a first embodiment in a refill position of the menstrual cup with replaceable absorbent liner.

FIG. 3 illustrates a top view of the first embodiment of the menstrual cup with replaceable absorbent liner.

FIG. 4 illustrates a cross-sectional view of the first embodiment in a use position of the menstrual cup with replaceable absorbent liner.

FIG. 5 illustrates a cross-sectional view of the first embodiment in a refill position of the menstrual cup with replaceable absorbent liner.

FIG. 6 illustrates a view of a single piece absorbent liner of the menstrual cup with replaceable absorbent liner.

FIG. 7 illustrates a view of a multi-piece absorbent liner of the menstrual cup with replaceable absorbent liner.

FIG. 8 illustrates a top view of a second embodiment in a use position of the menstrual cup with replaceable absorbent liner.

FIG. 9 illustrates an isometric view of a second embodiment in a use position of the menstrual cup with replaceable absorbent liner.

FIG. 10 illustrates an isometric view of a second embodiment in a refill position of the menstrual cup with replaceable absorbent liner.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

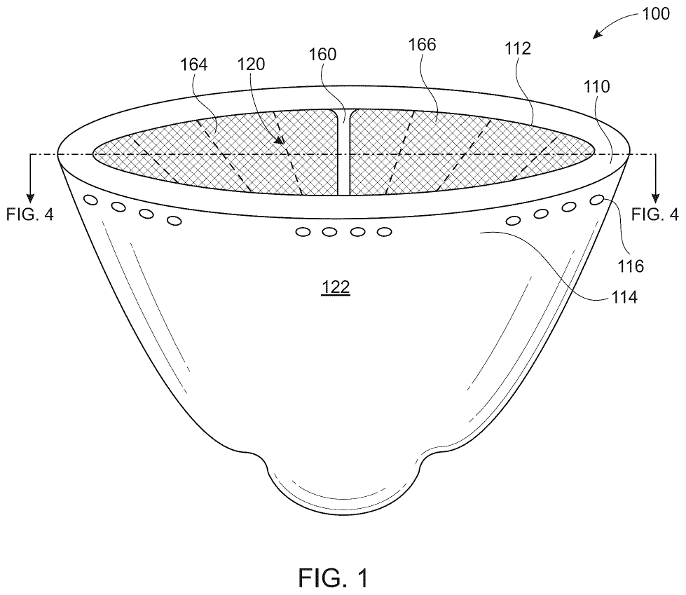

Referring to FIG. 1, an isometric view of the first embodiment in a use position of the menstrual cup with replaceable absorbent liner is shown.

The menstrual cup with replaceable absorbent liner 100 is shown in a first embodiment.

A reservoir 120 or body is topped with a rim 110, including a rim inner surface 112 and a rim outer surface 114 with optional rim outer projections 116.

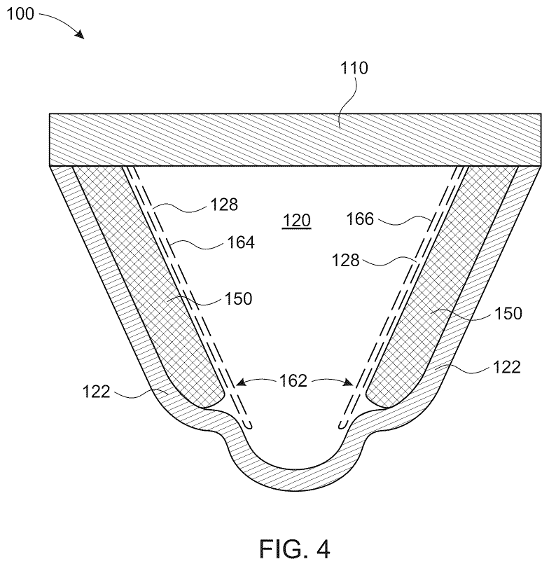

An exterior barrier 122 is impervious to fluids, therefore containing menstrual discharge within the reservoir 120, where it can be absorbed by the absorbent liner 150 (see FIG. 4).

The first embodiment includes a reservoir 120 with flexible internal walls 162. The flexible internal walls 162 include a first flap 164 and a second flap 166. Either flap 164/166 is accessed through, and separated by, the access opening 160, shown as a central slit or gap. The flaps 164/166 can be lifted by insertion of a finger into the access opening 160, allowing for replacement of the absorbent layer 150 (see FIG. 4).

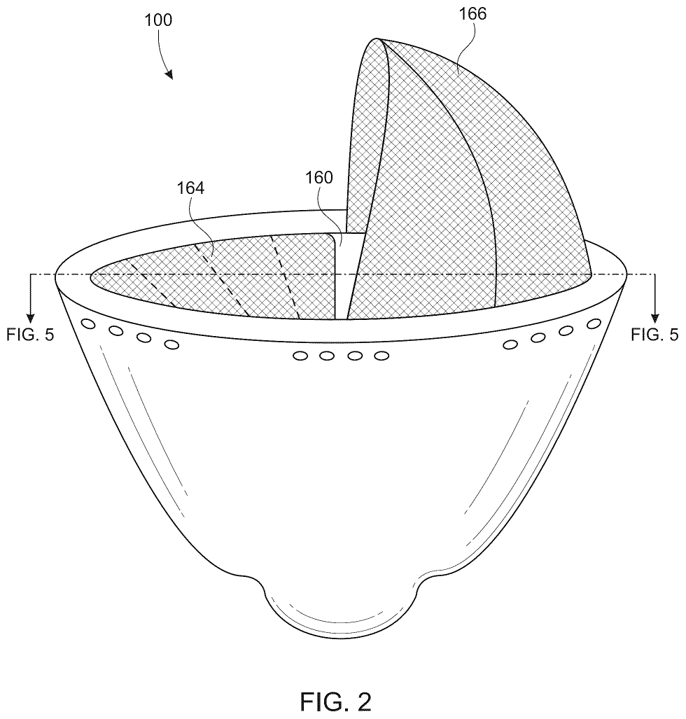

Referring to FIG. 2, an isometric view of a first embodiment in a refill position of the menstrual cup with replaceable absorbent liner is shown.

The menstrual cup with replaceable absorbent liner 100 is shown with the second flap 166 in an open position, allowing access to the inside for replacement of the absorbent liner 150.

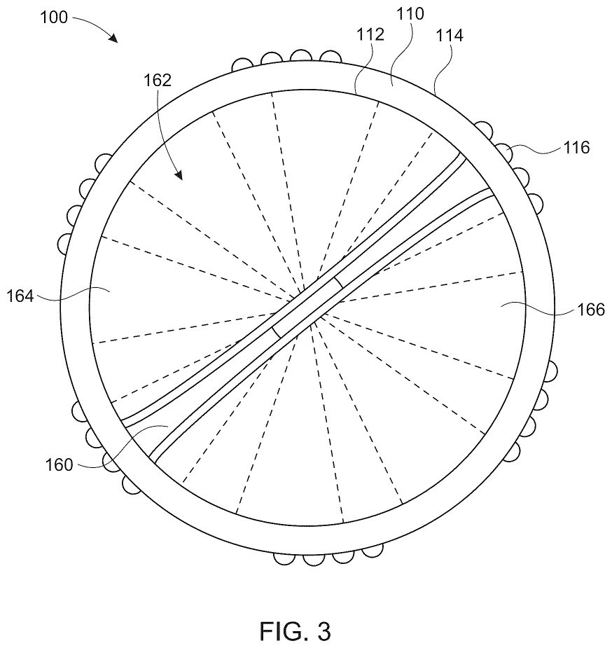

Referring to FIG. 3, a top view of the first embodiment of the menstrual cup with replaceable absorbent liner is shown.

The menstrual cup with replaceable absorbent liner 100 includes a rim 110 with rim inner surface 112 and rim outer surface 114 with optional rim outer projections 116. The flexible internal walls 162 are shown divided into a first flap 164 and a second flap 166. In alternative embodiments, there are additional flaps, further subdividing the flexible internal walls 162. In an additional alternative embodiment, there is a single flexible internal wall 162 and the access opening 160 is moved to a position along the rim 110, rather than being centrally located.

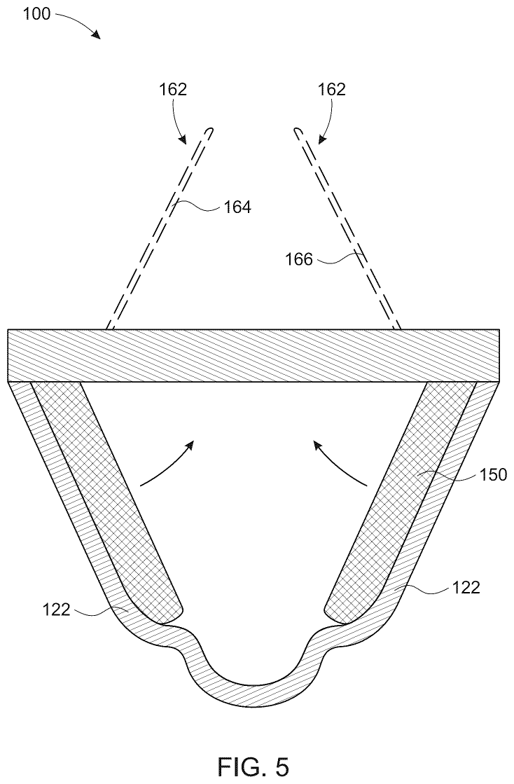

Referring to FIGS. 4 and 5, a cross-sectional view of the first embodiment in a use position and in a refill position, of the menstrual cup with replaceable absorbent liner is shown.

The menstrual cup with replaceable absorbent liner 100 is shown in both a use position and a position allowing for removal of the absorbent liners 150 for replacement.

Generally, the flexible internal walls 162 shown are divided into a first flap 164 and a second flap 166. The flaps 164/166 secure the absorbent liner 150 against the exterior barrier 122. Any menstrual fluid passes into the reservoir 120, through the perforations 128, and is absorbed into the absorbent liner 150.

When it is time to replace the absorbent liner 150, the flexible internal walls 162 are moved out of the way, allowing for removal of the absorbent liner 150, for replacement with a new absorbent liner 150. The flexible internal walls are then replaced, again maintaining the absorbent liner 150 in position for a subsequent use.

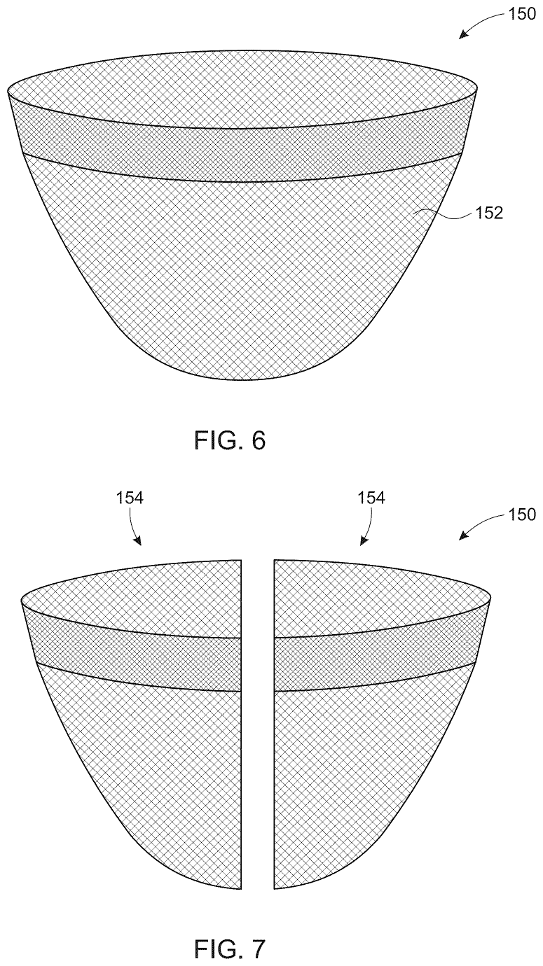

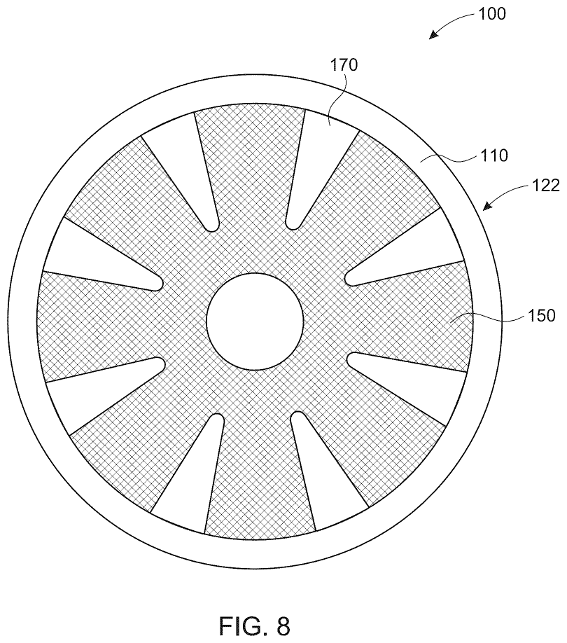

Referring to FIGS. 6 and 7, a view of a single piece absorbent liner and a view of a multi-piece absorbent liner, of the menstrual cup with replaceable absorbent liner, are shown.

The absorbent liner 150 can be a single-part absorbent liner 152 or a multi-part absorbent liner 154. The multi-part absorbent liner 154 is shown as two pieces but could also be formed from three or more pieces.

Referring to FIG. 8, a top view of a second embodiment in a use position of the menstrual cup with replaceable absorbent liner is shown.

In the second embodiment of the menstrual cup with replaceable absorbent liner 100, the flexible internal walls 162 (see FIG. 4) have been divided into multiple individual protrusions 170, which can also be referred to as fingers, resilient clamps, or hold-downs.

The individual protrusions 170 press the absorbent liner 150 against the exterior barrier 122, holding the absorbent liner 150 in place.

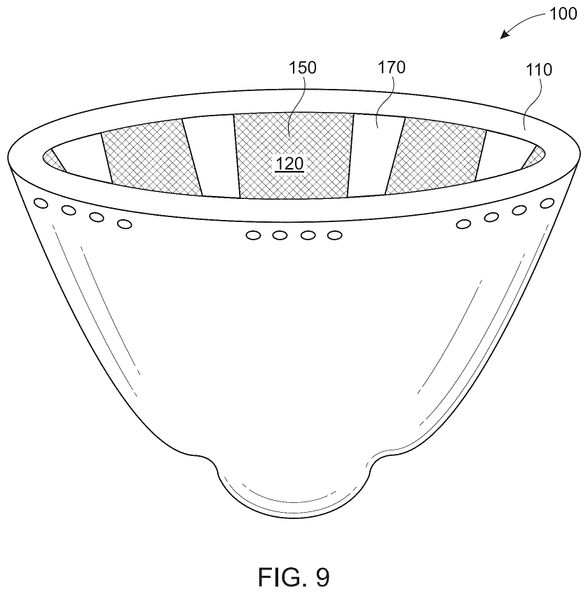

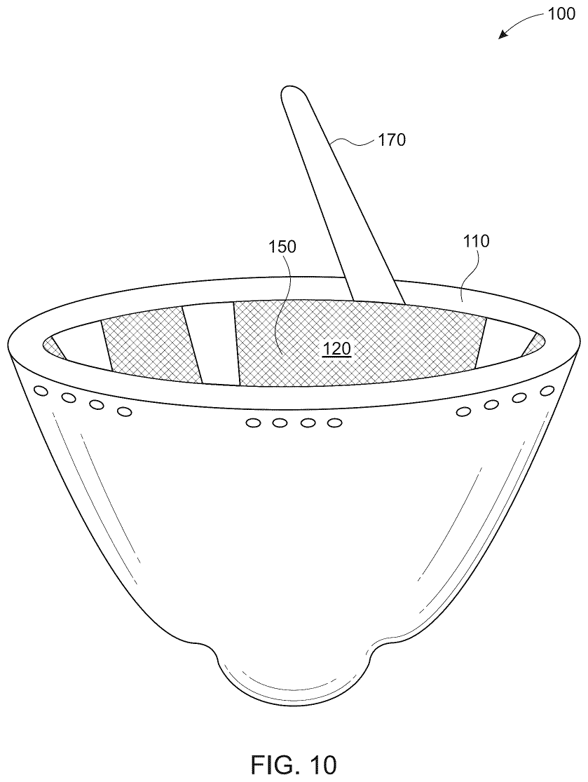

Referring to FIGS. 9 and 10, an isometric view of a second embodiment in a use position, and in a refill position, of the menstrual cup with replaceable absorbent liner are shown.

The individual protrusions 170 hold the absorbent liner in place within the reservoir 120. By being flexible, either via a hinge or construction with a flexible resilient material, each individual protrusion 170 may be lifted, allowing the user to access and replace the absorbent liner 150.

Equivalent elements can be substituted for the ones set forth above such that they perform in substantially the same manner in substantially the same way for achieving substantially the same result.

What is claimed is:

1. A menstrual cup with reusable reservoir and disposable absorbent layer, the menstrual cup comprising:
a reservoir defined by a rim and an external wall;
an absorbent layer;
an internal retaining mechanism;
the internal retaining mechanism coupled to the reservoir;
the internal retaining mechanism configured to removably hold the absorbent layer against an interior surface of the external wall;
wherein the absorbent layer is replaceable from an interior of the reservoir.

2. The menstrual cup with reusable reservoir and disposable absorbent layer of claim 1, wherein the internal retaining mechanism coupled to the rim of the reservoir.

3. The menstrual cup with reusable reservoir and disposable absorbent layer of claim 1, wherein the internal retaining mechanism is one or more flexible internal walls;
each flexible internal wall of the one or more flexible internal walls having a first position within the reservoir, and having a second position extended outside of the reservoir;
the absorbent layer able to be accessed by moving the one or more flexible internal walls to the second position;
whereby a position of the one or more flexible internal walls either holds the absorbent layer in place against the interior surface of the external wall, or allows for removal of the absorbent layer.

4. The menstrual cup with reusable reservoir and disposable absorbent layer of claim 3, wherein the one or more flexible internal walls is a first flap and a second flap;
the first flap separated from the second flap by an access opening;
the first flap able to be lifted by insertion of a finger into the access opening, allowing for replacement of the absorbent layer.

5. The menstrual cup with reusable reservoir and disposable absorbent layer of claim 1, wherein the internal retaining mechanism is multiple individual protrusions, the multiple individual protrusions acting to hold the absorbent layer within the reservoir.

6. The menstrual cup with reusable reservoir and disposable absorbent layer of claim 1, wherein in the absorbent layer is formed from a disposable material such as cotton.

7. A partially-reusable menstrual cup comprising:
a rim and external wall defining a reservoir;
an internal retaining mechanism;
the internal retaining mechanism configured to removably hold an absorbent layer against an interior surface of the external wall during use;
wherein the absorbent layer is replaceable from an interior of the partially-reusable menstrual cup.

8. The partially-reusable menstrual cup of claim 7, wherein the internal retaining mechanism coupled to the rim of the reservoir.

9. The partially-reusable menstrual cup of claim 7, wherein the internal retaining mechanism is one or more flexible internal walls;
each flexible internal wall of the one or more flexible internal walls having a first position within the reservoir, and having a second position extended outside of the reservoir;
the absorbent layer able to be accessed by moving the one or more flexible internal walls to the second position;
whereby a position of the one or more flexible internal walls either holds the absorbent layer in place against the interior surface of the external wall, or allows for removal of the absorbent layer.

10. The partially-reusable menstrual cup of claim 9, wherein the one or more flexible internal walls is a first flap and a second flap;
the first flap separated from the second flap by an access opening;
the first flap able to be lifted by insertion of a finger into the access opening, allowing for replacement of the absorbent layer.

11. The partially-reusable menstrual cup of claim 7, wherein the internal retaining mechanism is multiple individual protrusions, the multiple individual protrusions acting to hold the absorbent layer within the reservoir.

12. The partially-reusable menstrual cup of claim 7, wherein in the absorbent layer is formed from a disposable material such as cotton.

13. A menstrual cup device, comprising:
a reusable cup body having a rim and an external wall;
an internal structure coupled to the reusable cup body;
the internal structure configured to removably hold a disposable absorbent pad against an inner surface of the external wall;
wherein the disposable absorbent pad is replaceable by accessing an interior of the reusable cup body through the rim.

14. The menstrual cup device of claim 13, wherein the internal structure is coupled to the rim of the reusable cup body.

15. The menstrual cup device of claim 13, wherein the internal structure is one or more flexible internal walls;
each flexible internal wall of the one or more flexible internal walls having a first position within the reusable cup body, and having a second position extended outside of the reusable cup body;
the disposable absorbent pad able to be accessed by moving the one or more flexible internal walls to the second position;
whereby a position of the one or more flexible internal walls either holds the disposable absorbent pad in place against an interior surface of the external wall, or allows for removal of the disposable absorbent pad.

16. The menstrual cup device of claim 15, wherein the one or more flexible internal walls is a first flap and a second flap;
the first flap separated from the second flap by an access opening;
the first flap able to be lifted by insertion of a finger into the access opening, allowing for replacement of the disposable absorbent pad.

17. The menstrual cup device of claim 13, wherein the internal structure is multiple individual protrusions, the multiple individual protrusions acting to hold the disposable absorbent pad within the reusable cup body.

18. The menstrual cup device of claim 13, wherein the disposable absorbent pad is formed from a disposable material such as cotton.
